(19) United States
(12) Patent Application Publication
(10) Pub. No.: US 2015/0182172 A1
(43) Pub. Date: Jul. 2, 2015

(54) Systems and Methods Utilizing Plethysmographic Data for Distinguishing Arterial and Venous Oxygen Saturations

(71) Applicant: YALE UNIVERSITY, New Haven, CT (US)

(72) Inventors: Kirk H. Shelley, New Haven, CT (US); David G. Silverman, New Haven, CT (US); Zachary Doyle Walton, New Haven, CT (US)

(73) Assignee: YALE UNIVERSITY, New Haven, CT (US)

(21) Appl. No.: 14/559,721

(22) Filed: Dec. 3, 2014

Related U.S. Application Data
(62) Division of application No. 13/378,648, filed on Feb. 22, 2012, now abandoned.
(60) Provisional application No. 61/186,927, filed on Jun. 15, 2009.

(51) Int. Cl. A61B 5/00; A61B 5/1455

(52) U.S. Cl. CPC A61B 5/7278; A61B 5/14551

(57) ABSTRACT

Apparatus, systems and methods are provided for using the PG waveform to determine peripheral venous and arterial saturations. Generally, saturations are determined by isolating an indicator of venous or arterial blood volume in each of a plurality of PG waveforms and using the isolated indicators to determine saturation in the corresponding region of the vasculature. Indicators may include respiratory induced variations of AC and/or DC components of the PG waveforms, peaks of the PG waveforms, troughs of the PG waveforms, venous pulsations of the PG waveforms, etc. Indicators may further be isolated in either the time or frequency domain. The isolated indicators may advantageously be normalized, e.g., based on a baseline of the PG waveform or a derivative thereof.

### CROSS REFERENCE TO RELATED APPLICATIONS

The present application is a divisional patent application that claims priority benefit to a commonly-assigned and co-pending non-provisional patent application entitled “Systems and Methods Utilizing Plethysmographic Data For Distinguishing Arterial and Venous Saturations” (Ser. No. 13/378,648; filed Dec. 15, 2011), which was filed as a §371 national phase filing of PCT/US2010/038648, filed Jun. 15, 2010, which in turn claimed priority provisional patent application Ser. No. 61/186,927 filed on Jun. 5, 2009.

### BACKGROUND

1. Technical Field The present disclosure relates to systems and methods for studying and utilizing flow waveforms in the peripheral vasculature. In particular, the present disclosure relates to systems and methods for analyzing a plethysmograph (PG) waveform, as may be obtained using, e.g., a pulse oximeter, for facilitating calculation of arterial and venous saturation, e.g., oxygen saturation.

2. Background Information

The present disclosure is related to the subject matter of U.S. Patent Publication No. 2007/0032732 to Shelley et al., entitled “Method of Assessing Blood Volume using Photoelectric Plethysmography” (the “Shelley Patent Publication”). The Shelley patent Publication is hereby incorporated by reference, in its entirety.

The pulse oximeter has rapidly become one of the most commonly used patient monitoring systems both in and out of the operating room. This popularity is undoubtedly due to the pulse oximeter's ability to non-invasively monitor peripheral oxygen saturation as well as basic cardiac functions (e.g., heart rhythm). In addition, pulse oximeters are relatively easy to use and comfortable for the patient.

While the predominant application of a pulse oximeter has been calculating oxygen saturation of Hb, a pulse oximeter also inherently functions as a plethysmograph (more particularly, a photoplethysmograph), measuring minute changes in blood volume in a vascular bed (e.g., finger, ear or forehead), i.e., based on changes in light absorption. See, e.g., Hertzman, A B, “The Blood Supply of Various Skin Areas as Estimated By the Photoelectric Plethysmograph,” Am. J. Physiol. 124: 328-340 (1938). Thus, the raw plethysmograph (PG) waveform is rich in information relevant to the physiology of the patient. Indeed, the PG waveform contains a complex mixture of the influences of arterial, venous, autonomic and respiratory systems on the peripheral circulation.

A typical pulse oximeter waveform presented to a clinician, however, is a highly filtered and processed specter of the raw PG waveform. Indeed, it is normal practice for equipment manufacturers to use both auto-centering and auto-gain routines on the displayed waveforms so as to minimize variations in the displayed signal. While such signal processing may benefit certain calculations, it often comes at the expense of valuable physiological data. Thus, the greater potential of the raw PG waveform, remains largely overlooked.

Even when the raw PG waveform is considered and analyzed, it is often oversimplified. Indeed, the PG waveform is typically characterized as comprising two components: (i) a “pulsatile” (AC) component (traditionally attributed to variations in blood volume caused by the cardiac pulse) and (ii) a “non-pulsatile” (DC) component (traditionally attributed to “static” blood volume in nonpulsatile tissue, such as fat, bone, muscle and venous blood). It has since been demonstrated that the DC component of the PG waveform is, in fact, not “non-pulsatile” but, rather, is “weakly-pulsatile.” It has further been demonstrated that a number of physiological factors impact both the AC and DC components and that the PG waveform is far more complex than originally suspected.

In the Shelley patent publication, it was first noted that respiration/ventilation modulates both AC and DC components of a PG waveform. Thus, the Shelley patent publication disclosed, inter alia, apparatus, systems and methods for monitoring changes in blood volume by separating the impact of respiration/ventilation on the venous and arterial systems. More particularly, by isolating the impact of respiration/ventilation on predominantly arterial (AC) and predominantly venous (DC) components of the PG waveform one is able to independently assess changes in blood volume in different regions of the vasculature (arterial and venous). As noted in the Shelley patent publication, the degree of respiratory-induced variation of the AC component of the PG waveform corresponds to arterial blood volume (more particularly, cardiac stroke volume). Similarly, as noted in the Shelley patent publication, the degree of respiratory-induced variation of the DC component of the PG waveform corresponds to effective venous blood volume.

Physiologically, changes in venous blood volume often correspond to changes in end-diastolic volume (EDV), i.e., the volume of blood in the ventricles after diastole. More particularly, venous blood volume and venous compliance (e.g., relating to venous tone) affect venous blood pressure and the rate of venous return which in turn impact EDV. Thus, activation of the baroreceptor reflex, such as during acute hemorrhaging, causes venoconstriction which results in decreased venous compliance, improved venous return, and increased end-diastolic volume. Similarly, changes in arterial blood volume correspond to cardiac stroke volume, i.e., the difference between EDV and end-systolic volume (ESV). Cardiac output is determined as cardiac stroke volume multiplied by heart rate. Notably venous compliance is significantly (20-24 times) greater than arterial compliance.

One method suggested by the Shelley patent publication for extracting and analyzing impact of respiration/ventilation on the venous and arterial systems includes comparing tracings of the peaks and valleys of the PPG waveform. Thus, respiratory-induced variation of the of the AC and DC components may be isolated, e.g., based on the amplitude and the average of the PG wavefrom, respectively.

AC and DC components of a PG waveform may also be isolated by applying active frequency filters during sampling (the signal from the photodetector may be time demultiplexed such that each frequency can be processed independently).

Another method suggested by the Shelley patent publication for assessing changes in blood volume involves harmonic analysis, e.g., Fourier analysis, of the PG waveform. Harmonic analysis allows for the extraction of underlying signals that contribute to a complex waveform. As disclosed in the Shelley patent publication, harmonic analysis of the PG waveform principally involves a short-time Fourier transform of the PG waveform. In particular, the PG waveform may be converted to a numeric series of data points via analog to digital conversion, wherein the PG waveform is sampled at a predetermined frequency, e.g., 50Hz, over a given time period, e.g., 60-90 seconds. A Fourier transform may then be performed on the data set in the digital buffer (note that the sampled PG waveform may also be multiplied by a windowing function, e.g., a Hamming window, to counter spectral leakage). The resultant data may further be expanded in logarithmic fashion, e.g., to account for the overwhelming signal strength of the cardiac frequencies relative to the ventilation frequencies. It is noted that while the Shelley patent publication discloses using joint time-frequency analysis, i.e., a spectrogram, as a preferred technique for viewing and analyzing spectral density estimation of the PG waveform, a spectrum for the PG waveform, as used herein, may be extrapolated therefrom for any discrete sampling period.

According to the Shelley patent publication, PG waveform analysis, such as described above, may be used to independently monitor changes in arterial and venous blood volume. For instance, respiratory induced variation of the AC component, represented in the frequency-domain as side-band modulation around the cardiac signal, is indicative of changes in blood volume severe enough to affect cardiac output. Similarly, increased respiratory-induced variation of the DC component of a PG waveform, represented in the frequency domain as an increase in signal strength for the respiratory signal, is indicative of venous loss (it is noted however that decreased cardiac output may also, at times, contribute to changes in the respiratory signal). Thus, by monitoring side-band modulation of the cardiac signal, one is able detect changes in cardiac output and arterial blood volume. Similarly, by monitoring variations in the respiratory signal, one is able to detect changes in effective venous blood volume.

In medicine, oxygen saturation (SO₂), measures the percentage of oxygenated hemoglobin (HbO₂) relative to the total number of hemoglobin (Hb) molecules. As mentioned above, pulse oximetry provides a simple non-invasive method for monitoring oxygen saturation in the peripheral vasculature (SpO₂). A basic pulse oximeter includes a probe that is brought into contact with a patient, e.g., by way of attachment to a patient's finger, ear, forehead, etc., which is linked to a computerized unit for processing. A source of light originates from the probe at two wavelengths, e.g., 660 nm (Red) and 940 nm (IR). The light is partly absorbed by hemoglobin, and the absorption level differs from wavelength-to-wavelength depending on the degree of oxygen saturation. Beer's law (the Beer-Lambert or Bouguer-Beer relation) provides that there exists a inverse logarithmic dependence between the absorbance of light through a substance and the product of the concentration of the absorbing species in the material and the distance the light travels through the material (i.e. the path length). Thus, by monitoring absorption at each of the wavelengths, one is able to approximate SpO₂. In general, for a given pulse oximeter signal, SpO₂ may be calculated using a ratio R=Red/IR. A common approximation of SpO₂ is calculated as: SpO₂=110-25 R.

Peripheral arterial oxygen saturation SpₐO₂ is typically calculated using an arterial Red/IR ratio (Rₐ) of ratios of light absorption between the AC component of the PG waveform and the DC component of the PG waveform, at two or more wavelengths: Rₐ=(Red AC/Red DC)/(IR AC/IR DC). In this way, the pulsatile component of the PG waveform is, in effect, normalized relative to the nonpulsatile or weakly pulsatile component of the PG waveform.

Despite advancements to date a need exists for improved apparatus, systems and methods for assessing oxygen saturation in different regions of the vasculature. These and other needs are satisfied by the apparatus, systems and methods of the present disclosure.

### SUMMARY

Various apparatus, systems and methods are described herein for determining saturation, e.g., oxygen saturation, in a particular vascular region.

In one exemplary embodiment, venous saturation is determined by (i) detecting a PG waveform for each of a plurality of wavelengths; (ii) determining an amplitude of respiratory induced variation of a DC component for each of the detected PG waveforms, wherein the amplitude of respiratory induced variation is normalized relative to a baseline of the DC component; and (iii) calculating a venous saturation corresponding to a set of all the determined amplitudes.

In another exemplary embodiment, venous saturation is determined by (i) detecting a plethymograph (PG) waveform for each of a plurality of wavelengths; (ii) isolating venous pulsations for each of the detected PG waveforms and (iii) calculating a venous saturation based on the isolated venous pulsations.

In another exemplary embodiment, venous saturation is determined by (i) detecting a plethymograph (PG) waveform for each of a plurality of wavelengths; (ii) isolating troughs for each of the detected PG waveforms and (iii) calculating a venous saturation based on the isolated troughs.

In another exemplary embodiment, arterial saturation is determined by (i) detecting a plethymograph (PG) waveform for each of a plurality of wavelengths; (ii) determining an amplitude of respiratory induced variation of an AC component for each of the detected waveforms and (iii) calculating an arterial saturation corresponding to a set of all the determined amplitudes.

In another exemplary embodiment, arterial saturation is determined by (i) detecting a plethymograph (PG) waveform for each of a plurality of wavelengths; (ii) isolating peaks for each of the detected PG waveforms and (iii) calculating an arterial saturation based on the isolated peaks.

In another exemplary embodiment, arterial saturation is determined by (i) detecting a plethymograph (PG) waveform for each of a plurality of wavelengths; (ii) isolating a cardiac signal in the frequency domain for each of the detected PG waveforms, wherein the isolated cardiac signal is normalized based on signal strength at the ultra-low frequencies and (iii) calculating a venous saturation based on the isolated cardiac signals.

In another exemplary embodiment, saturation in a particular vascular region is determined by (i) detecting a plethymograph (PG) waveform for each of a plurality of wavelengths; (ii) calculating an instantaneous saturation for the detected PG waveforms and (iii) extrapolating venous saturation or arterial saturation based on changes in the instantaneous saturation.

In another exemplary embodiment, oxygen saturation in a particular vascular region is determined by (i) detecting a plethymograph (PG) waveform for each of red and infrared wavelengths; (ii) plotting red vs. infrared PG values on a graph to form two lobes and (iii) extrapolating a venous oxygen saturation or an arterial oxygen saturation based on a slope of one of the lobes.

Apparatus and systems generally comprise a probe and/or a processor adapted to execute the methods described herein.

Additional features, functions and benefits of the disclosed apparatus, systems and methods will be apparent from the description which follows, particularly when read in conjunction with the appended figures.

### BRIEF DESCRIPTION OF THE DRAWINGS

To assist those of ordinary skill in the art in making and using the disclosed apparatus, systems and methods, reference is made to the appended figure, wherein:

FIG. 1 depicts isolation of peaks and valleys of a PG waveform and extraction of tracings thereof, according to the present disclosure.

FIG. 2 depicts the isolation of the respiratory effect on AC and DC components of a PG signal, according to the present disclosure.

FIG. 3 depicts pulsatile and nonpulsatile components of an extracted respiratory effect on the DC component of a PG waveform, according to the present disclosure.

FIG. 4 depicts arterial and venous pulsations and troughs of a PG waveform, according to the present disclosure.

FIG. 5 depicts AC and DC components of a PG waveform extracted using active filtration during sampling, according to the present disclosure.

FIG. 6 depicts arterial and venous components as reflected in a PG spectrum, according to the present disclosure.

FIG. 7 depicts time variant and time in-varient components of the DC component of a PG waveform as reflected in the frequency domain, according to the present disclosure.

FIG. 8 depicts a Red vs. IR plot of a PG waveform having arterial and venous lobes.

FIG. 9 depicts a normalized Red vs. IR plot of a PG.

FIG. 10 depicts a frequency domain representation of exemplary Red/IR PG waveforms including an oxygen saturation overlay.

### DESCRIPTION OF EXEMPLARY EMBODIMENT(S)

The present disclosure expands on the known usefulness of the PG waveform. In particular, the present disclosure relates to improved apparatus, systems and methods for using the PG waveform to determine peripheral venous and arterial saturations, e.g., oxygen saturations. Thus, in clinical applications, while a decreasing or low arterial oxygen saturation, e.g., below 90%, is indicative of hypoxemia, a decreasing or low venous oxygen saturation, e.g., below 60%, may be indicative of altered tissue factors, such as inadequate tissue perfusion or excessive tissue oxygen consumption, as well as hypoxemia; and an excessively high venous oxygen saturation, e.g., approximating the arterial value, may be indicative of arteriovenous shunting without capillary tissue exchange. As is universally known, mixed venous saturation, measured at the pulmonary artery is an excellent indicator of tissue perfusion adequacy on a global level throughout the body. According to the present disclosure peripheral venous oxygen saturation may serve as early indicator of impending changes in mixed venous saturation.

Various apparatus systems and methods are described herein for extracting arterial and venous components of the PG waveform in both time and frequency domains. As disclosed herein, the ability to isolate each of the pulsatile (arterial) and nonpulsatile or weakly pulsatile (venous) components of the PG waveform enables one to independently assess saturation, e.g., oxygen saturation, in two different regions of the vasculature (arterial and venous). It is noted that the various apparatus, systems and methods presented herein for determining peripheral venous and arterial oxygen saturation may be employed independently or in conjunction with one another. Thus, for example one technique may be used to provide calibration data for or to cross-check the veracity of readings obtained using another technique.

Time Domain Analysis

As noted above, one method (in the time domain) for extracting arterial and venous components of the PG waveform is to isolate the effects of respiration on both AC and DC components of the PPG waveform. More particularly, the effect of respiration on the AC component of the PG waveform may be determined, e.g., by calculating changes in the amplitude of the PG waveform. Similarly, the effect of respiration on the DC component of the PG waveform may be determined by calculating changes in the baseline of the PG waveform.

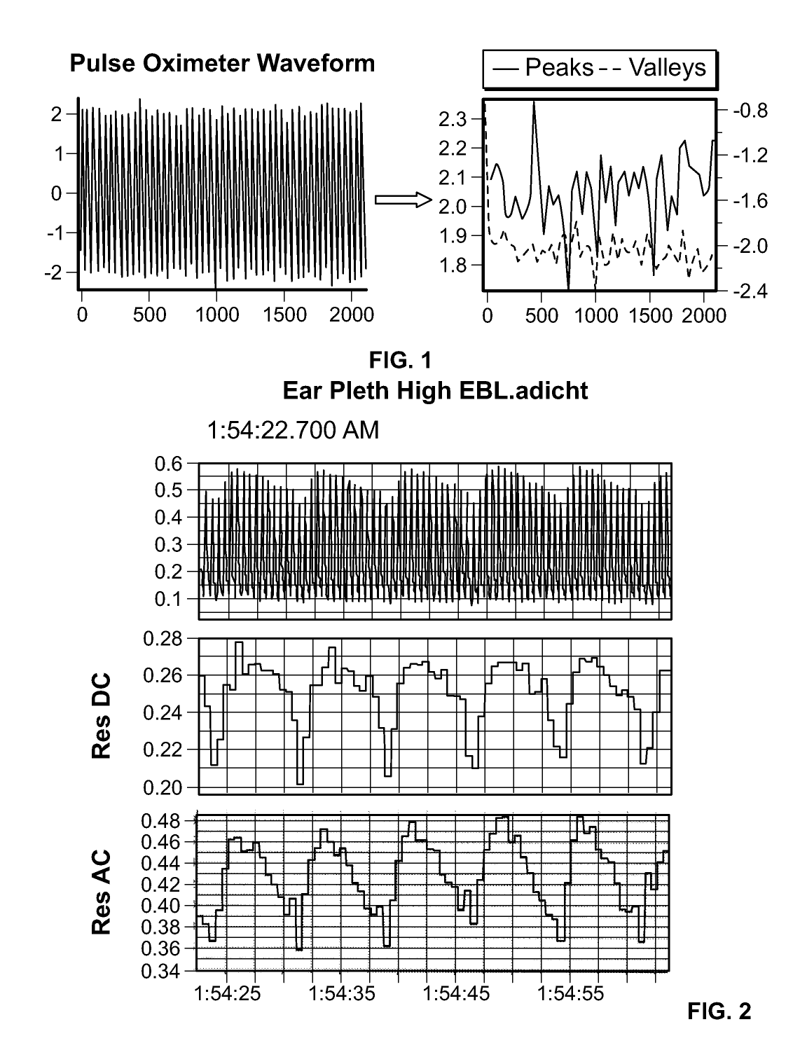

Referring now to FIGS. 1 and 2, the effects of respiration on each of the AC and DC components of the PG waveform may be estimated, in the time domain, using tracings of the peaks and valleys of the PG waveform. More particularly, the effect of respiration on the AC component of the PG waveform (“ResAC”) may be approximated, e.g., by subtracting the tracing of the valleys from the tracing of the peaks and dividing the result by 2. Similarly, the effect of respiration on the DC component of the PG waveform (“ResDC”) may be approximated, e.g., by averaging the two tracings.

According to the present disclosure, the respiratory effect on AC and/or DC components may be determined, e.g., for a pair PG waveforms at different wavelengths (Red and IR). Thus, in exemplary embodiments, the peripheral venous oxygen saturation (SpᵥO₂) may be determined, e.g., using a venous Red/IR ratio (Rᵥ) calculated by dividing the tracing of the respiratory effect on the DC component (ResDC) (Red) by ResDC (IR). Similarly, the peripheral arterial oxygen saturation (SpₐO₂) may be determined e.g., using an arterial Red/IR ratio (Rₐ) calculated by dividing the tracing of the respiratory effect on the AC (ResAC) (Red) by ResAC (IR). In exemplary embodiments, it may be advantageous to normalize each of ResDC and ResAC. By normalizing the extracted ResDC and ResAC, factors such as background absorption and variations in the path lengths of light, may be advantageously accounted for. Normalization may be achieved by calculating a ratio of ratios using pulsatile and non-pulsatile components of the extracted ResDC or ResAC.

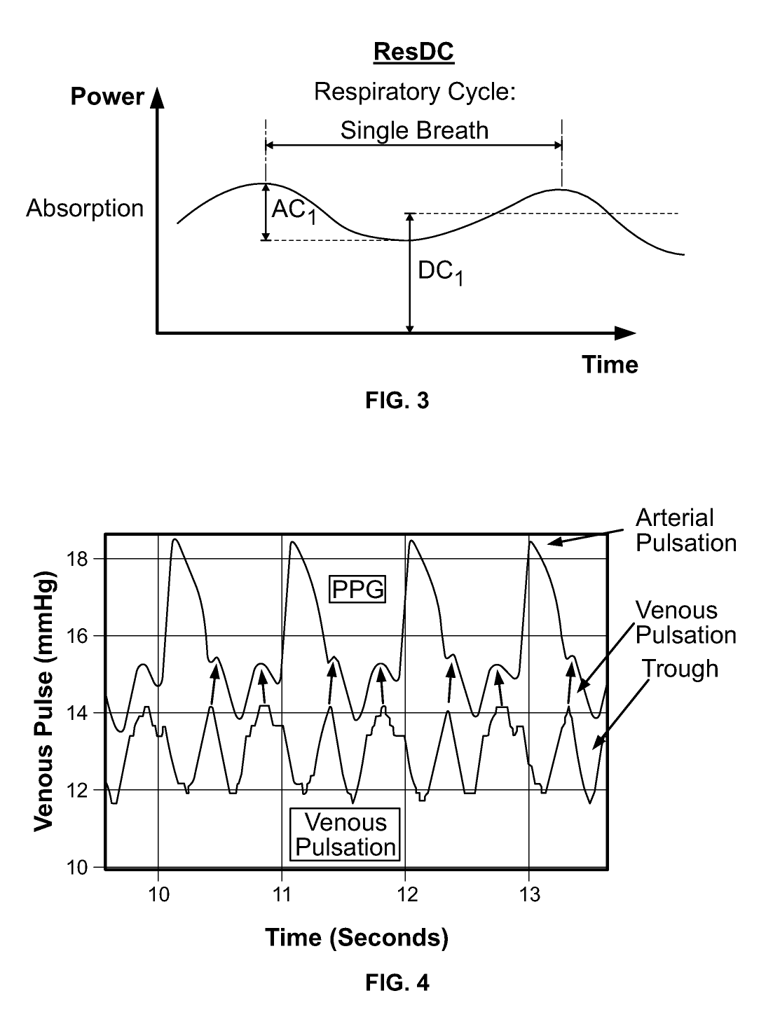

Referring now to FIG. 3, an exemplary ResDC is depicted. The exemplary ResDC, includes both a time-variant component at the respiratory frequency (AC₁), e.g., due to the effect of respiration on venous blood volume and an offset (DC₁), e.g., due to background absorption. Similarly, the ResAC tracing of a PG waveform may include both a time-variant component at the respiratory frequency (AC₂), e.g., due to the effect of respiration on arterial blood volume and an offset (DC₂), e.g., due to background absorption. Thus, according to the present disclosure, a venous Red/IR ratio (Rᵥ) of ratios may be calculated as:

Rᵥ=(AC₁Red/DC₁Red)/(AC₁IR/DC₁IR).

Similarly, an arterial Red/IR ratio (Rₐ) of ratios may be calculated as:

Rₐ=(AC₂Red/DC₂Red)/(AC₂IR/DC₂IR).

Alternatively, the time-variant components of ResAC and ResDC may be normalized, based on the offset of the PG waveform. E.g., a venous Red/IR ratio (Rᵥ) may be calculated as:

Rᵥ=(AC₁Red/DC Red)/(AC₁IR/DC IR).

Similarly, an arterial Red/IR ratio (Rₐ) of ratios may be calculated as:

Rₐ=(AC₂Red/DC Red)/(AC₂IR/DC IR).

In further exemplary embodiments, arterial and venous components of the PG waveform may also be extracted by isolating arterial and venous pulsations of the PG waveform. FIG. 4 depicts an exemplary PG waveform including both arterial and venous pulsations (the actual venous pulse (mmHg) is also depicted). Thus, arterial and venous pulsations may be isolated for each of a pair PPG waveforms at different wavelengths (Red and IR), e.g., using a peak detection algorithm. The peripheral arterial oxygen saturation (SpₐO₂) may be determined, e.g., using an arterial Red/IR ratio (Rₐ) calculated by dividing the absorption value at the time of the arterial pulsation (AbsAP) (Red) by the AbsAP (IR). Similarly, the peripheral venous oxygen saturation (SpᵥO₂) may be determined, e.g., using a venous Red/IR ratio (Rᵥ) calculated by dividing absorption value at the time of the venous pulsation (AbsVP) (Red) by the AbsVP (IR).

It is further noted that the lowest point of PG waveform for each cardiac cycle (referred to herein as the “trough”) is highly indicative of venous activity. Thus, it is contemplated that such troughs may be isolated for each of a pair PPG waveforms at different wavelengths (Red and IR), wherein the peripheral venous oxygen saturation (SpᵥO₂) may be determined, e.g., using a venous Red/IR ratio (Rᵥ) calculated by dividing absorption value at the time of the trough (AbsTrough) (Red) by the AbsTrough (IR).

In exemplary embodiments, AbsAP, AbsVP and AbsTrough may be normalized, e.g., based on the offset of the PG waveform. Thus, according to the present disclosure, an arterial Red/IR ratio (Rₐ) of ratios may be calculated as:

Rₐ=(AbsAPRed/offsetRed)/(AbsAPIR/offsetIR).

Similarly, using the venous pulsations as a venous indicator, a venous Red/IR ratio (Rᵥ) of ratios may be calculated as:

Rᵥ=(AbsVPRed/offsetRed)/(AbsVPIR/offsetIR).

Using the troughs as a venous indicator, a venous Red/IR ratio (Rᵥ) of ratios may be calculated as:

Rᵥ=(AbsTroughRed/offsetRed)/(AbsTroughIR/offsetIR).

Alternatively, AbsAP, AbsVP and AbsTrough may be normalized based on pulsatile and non-pulsatile components derived from tracings of AbsAP, AbsVP and AbsTrough. This technique mirrors that disclosed with respect to ResAC and ResDC.

In exemplary embodiments, the PG waveform may be filtered, e.g., in the frequency domain, to isolate or exclude components associated with the venous/arterial components. For example, the PG waveform may be filtered to isolate the cardiac pulse (an arterial indicator), e.g., by extracting data in the cardiac frequencies (i.e., 0.75 to 3.0 Hz). The extracted data may then be analyzed in either the time domain or frequency domain and venous/arterial oxygen saturation may be determined, e.g., according to the apparatus, systems and methods provided herein or via a simple comparison of Red vs. IR absorption values for the frequency filtered data.

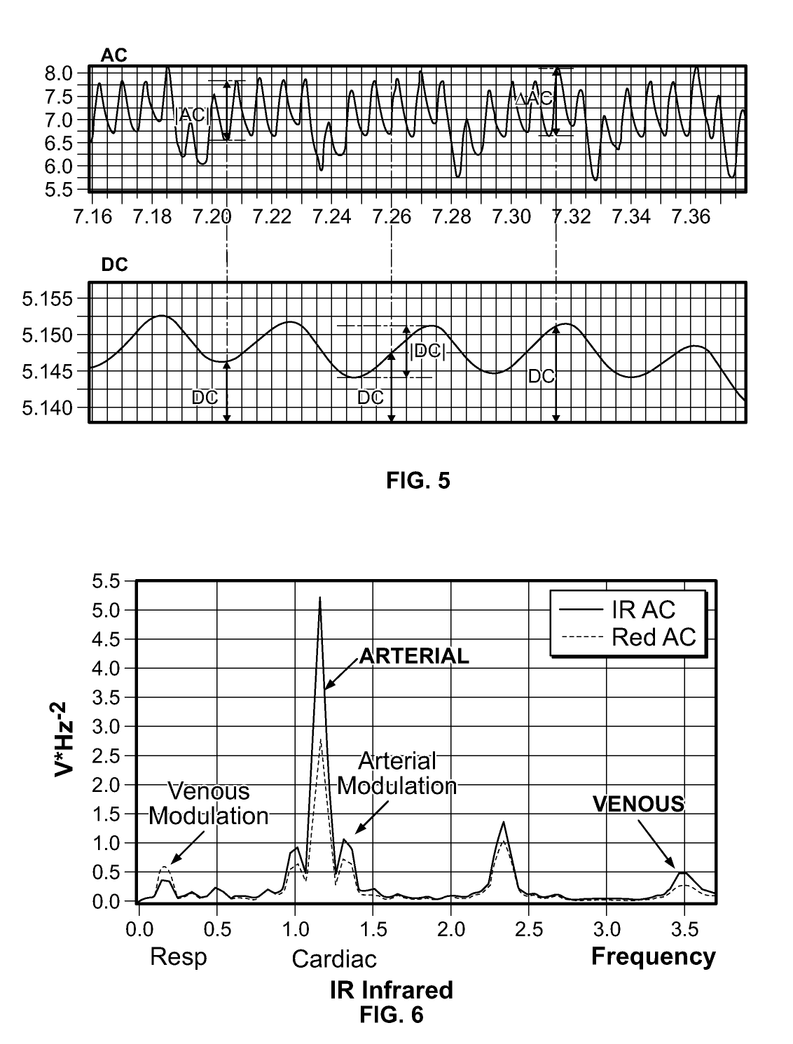

In further exemplary embodiments, arterial and venous components of the PG waveform may be extracted using active filtration during sampling to separate out AC and DC components of the PG waveform. Thus, e.g., frequencies below 0:45 Hz may be concentrated in the DC signal and frequencies above 0:45 Hz in the AC signal. FIG. 5 depicts AC and DC components of a PG waveform as extracted using active frequency filtration. Notably, any baseline modulation of the extracted AC component is most likely due to filter bleed-through.

Referring still to FIG. 5, an arterial Red/IR ratio of ratios (Rₐ) may be calculated as the using the peak-to-peak amplitude of the AC waveform (|AC|) normalized by the DC offset of the PG waveform (DC):

$${R}_{a} = \frac{{( \vert AC \vert \text{/} DC )}_{Red}}{{( \vert AC \vert \text{/} DC )}_{IR}}$$

Similarly, a venous Red/IR ratio of ratios (Rᵥ) may be calculated as the using the peak-to-peak amplitude of the extracted DC waveform (|DC|) normalized by the DC offset of the PG waveform (DC):

$${R}_{v} = \frac{{( \vert DC \vert \text{/} DC )}_{Red}}{{( \vert DC \vert \text{/} DC )}_{IR}}$$

In exemplary embodiments, an instantaneous oxygen saturation may be calculated for the PG waveform. Thus, venous oxygen saturation may be detected, e.g., by monitoring changes in the minimum oxygen saturation value or a lower range of oxygen saturation values over a cardiac cycle. Similarly, arterial oxygen saturation may be detected, e.g., by monitoring changes in the maximum oxygen saturation value or an upper range of oxygen saturation values over a cardiac cycle.

In further exemplary embodiments, an instantaneous saturation waveform may be extracted from the AC and DC components by calculating a ratio of ratios R using the value of the AC waveform minus the waveform minimum (ΔAC) normalized by the DC offset of the PG signal:

R=((ΔAC)/CD)_{Red}/((ΔAC)/CD)_{IR}

In exemplary embodiments, the waveform minimum is defined as the waveform value at the preceding trough.

One complication of the instantaneous saturation waveform method is the inherent instability near the troughs of the AC waveform. As noted above, both the numerator and the denominator of R are proportional to changes in the AC waveform relative to the minimum value of AC, e.g., over the preceding cardiac cycle. Thus, in the vicinity of the troughs, both the numerator and denominator of R approach zero, and the overall fraction becomes unstable. To address this instability, a threshold feature may be applied. Thus, in exemplary embodiments, the difference between each AC waveform data point and the preceding trough is compared to the DC offset. If the ratio of these two quantities is less than a threshold value, e.g., of 3%, the saturation calculated using these values is discarded and the saturation from the previous time is carried forward until the change in the AC waveform exceeds, e.g., 3% of the DC offset. The 3% threshold value is particularly advantageous since it prevents waveform instability while at the same time not ‘over-smoothing” the waveform.

Conceptually, thresholding can be thought of as applying a signal-to-noise cutoff. As the change in PG waveform approaches zero, the corresponding volume of blood in motion also approaches zero. Since the algorithm for calculating saturation inherently depends on blood in motion, the algorithm fails during the time periods when the blood is not moving. Fortunately, it may be assumed that the saturation of the blood in each compartment is approximately constant during these relatively short time periods.

Thresholding introduces artifacts into the instantaneous saturation waveform near where the change in the AC waveform crosses 3% of the DC offset. Therefore, a smoothing procedure may be implemented to replace each value with the mean of all of the values within a given time frame, e.g., 0:05 s of the point in question.

The instantaneous saturation waveform is a pulsatile waveform with peaks and valleys approximately coinciding with the peaks and valleys in the AC waveform. Thus, to obtain separate information about the arterial and the venous saturation, the peaks and valleys of the instantaneous saturation waveform are isolated, wherein the peaks correspond to arterial saturation and the valleys correspond to venous saturation.

Frequency Domain Analysis

Apparatus, systems and methods are also provide for calculating oxygen saturation for arterial and venous components of the PG waveform using harmonic analysis, e.g., Fourier analysis. As disclosed in the Shelley publication, harmonic analysis of the PG waveform principally involves a short-time Fourier transform of the PG waveform. In particular, the PG waveform may be recorded as a digital signal or, if analogue, converted to a numeric series of data points via analog to digital conversion, wherein the PG waveform is sampled at a predetermined frequency, e.g., 50 Hz, over a given time period, e.g., 60-90 seconds. A Fourier transform may then be performed on the data set in the digital buffer (note that the sampled PG waveform may also be multiplied by a windowing function, e.g., a Hamming window, to counter spectral leakage). The resultant data may further be expanded in logarithmic fashion, e.g., to account for the overwhelming signal strength of the cardiac frequencies relative to the ventilation frequencies. While the Shelley publication discloses joint time-frequency analysis, i.e., a spectrogram, as a preferred technique for viewing and analyzing spectral density estimation of the PG waveform, the spectrum of the PG waveform over a set period of time may be easily extrapolated therefrom.

An exemplary PG spectrum is depicted in FIG. 6. As previously mentioned, the Shelley publication disclosed, for the first time, that respiration/ventilation modulates both the AC and DC components of the PG waveform. Thus, according to the Shelley publication, harmonic analysis, such as described above, may be used to isolate the effects of respiration on both AC and DC components of the PG waveform, as reflected in the PG waveform spectrum. For example, changes in the PG waveform spectrum at or around the respiratory frequency (i.e. the respiratory signal) are observed to be principally reflective of ResDC. Similarly, side-band modulation around the primary band of the cardiac signal are correlated to ResAC.

In exemplary embodiments, the respiratory signal and/or side-bands of the cardiac signal may be isolated for each of a pair PG waveforms at different wavelengths (Red and IR), e.g., using a peak detection algorithm, calculating inflection points, using regression modes, etc. Thus, the peripheral arterial oxygen saturation (SpₐO₂) may be determined, e.g., using an arterial Red/IR ratio (Rₐ) calculated by dividing the signal strength (e.g., peak signal strength, area under the curve, root-mean-square, etc.) of one of the side-bands (or the average amplitude of the side-bands) (StengthSB) (Red) by the StrengthSB (IR). Similarly, the peripheral venous oxygen saturation (SpᵥO₂) may be determined, e.g., using a venous Red/IR ratio (Rᵥ) calculated by dividing the signal strength of the respiratory signal (StrengthRS) (Red) by the StrengthRS (IR).

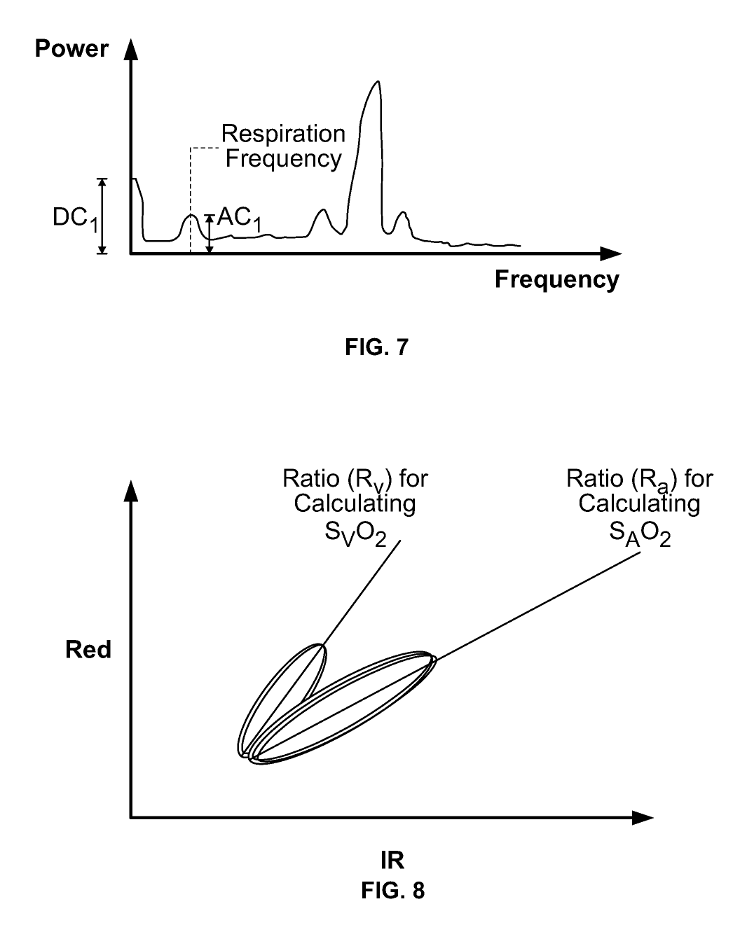

In further exemplary embodiments, StrengthRS and StrengthSB may be normalized. As discussed within the context of time domain analysis, ResDC may include both amplitude modulation (AC₁), e.g., due to the effect of respiration on venous blood volume, and an offset (DC₁), e.g., due to background absorption. As depicted in FIG. 7, in the frequency domain, the time variant component of ResDC (AC₁) is embodied at the respiratory frequencies and the time in-variant component of ResDC (DC₁) is embodied at the ultra-low frequencies. Thus, similar to normalization in the time domain, a venous Red/IR ratio (Rᵥ) of ratios may be calculated as:

Rᵥ=(AC₁Red/DC₁Red)/(AC₁IR/DC₁IR).

It is contemplated that, for frequency domain analysis, StrengthSB may also be normalized using the signal strength at the ultra-low frequencies. Alternately, it is contemplated that StrengthSB and/or StrengthRS may be normalized relative to the primary band of the cardiac signal.

According to the present disclosure, the primary band of the cardiac signal is primarily representative of arterial pulsations. Thus, it is contemplated that arterial oxygen saturation (SpₐO₂) may also be determined, e.g., using an arterial Red/IR ratio (Rₐ) calculated by dividing the strength of the primary band of the cardiac signal (StrengthPB) (Red) by the StrengthPB (IR). Similarly, upper harmonics of the cardiac signal are primarily representative of venous pulsations. Thus, it is contemplated that arterial oxygen saturation (SpₐO₂) may also be determined, e.g., using a venous Red/IR ratio (Rᵥ) calculated by dividing the strength of one of upper harmonics (UH) of the cardiac signal (StrengthUH) (Red) by the StrengthUH (IR). Note that StrengthPB and StrengthUH may be normalized in the same manner as disclosed with respect to StrengthSB and StrengthRS.

Calculating Red/IR Ratios Using Red Vs. IR Plots

In exemplary embodiments, as depicted in FIG. 8, it is contemplated that Rₐ and Rᵥ may be calculated by plotting corresponding absorption values for Red and IR PG waveform, relative to one another, over a given period of time, e.g., 60-90 sec. The resulting graph may include a two lobes, wherein a regression model may be applied to determine a slope value for each of the lobes, the greater slope value corresponding to Rᵥ and the lesser slope value corresponding to Rₐ.

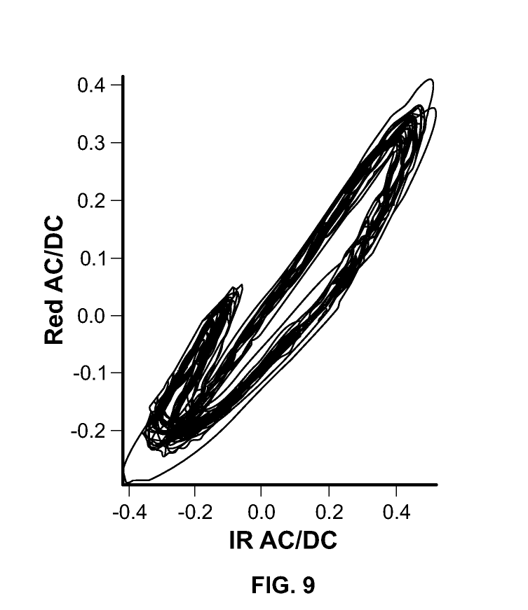

In exemplary embodiments, as depicted in FIG. 9, the Red vs. IR absorption values may be normalized, e.g., by dividing, for each PG waveform, the AC component of the PG waveform by the DC component of the PG waveform.

It is further contemplated that above method of calculating a Red/IR ratio may be applied with respect to any extracted/isolated venous and/or arterial component of the PG waveform. Thus, Rᵥ may be determined for any Red vs. IR absorption values plotted for one of (i) ResDC, (ii) AbsVP, (iii) AbsTough, (iv) StrengthRS and (v) StrengthUH. Similarly, Rₐ may be determined for any Red vs. IR absorption values plotted for one of: (i) ResAC, (ii) AbsAP, (iii) StrengthSB and (iv) StrengthPB. It is noted that the plotted absorption values for any of the above extracted/isolated venous and/or arterial components may or may not be normalized in accordance with the methods disclosed herein.

Graphic Overlay of Oxygen Saturation

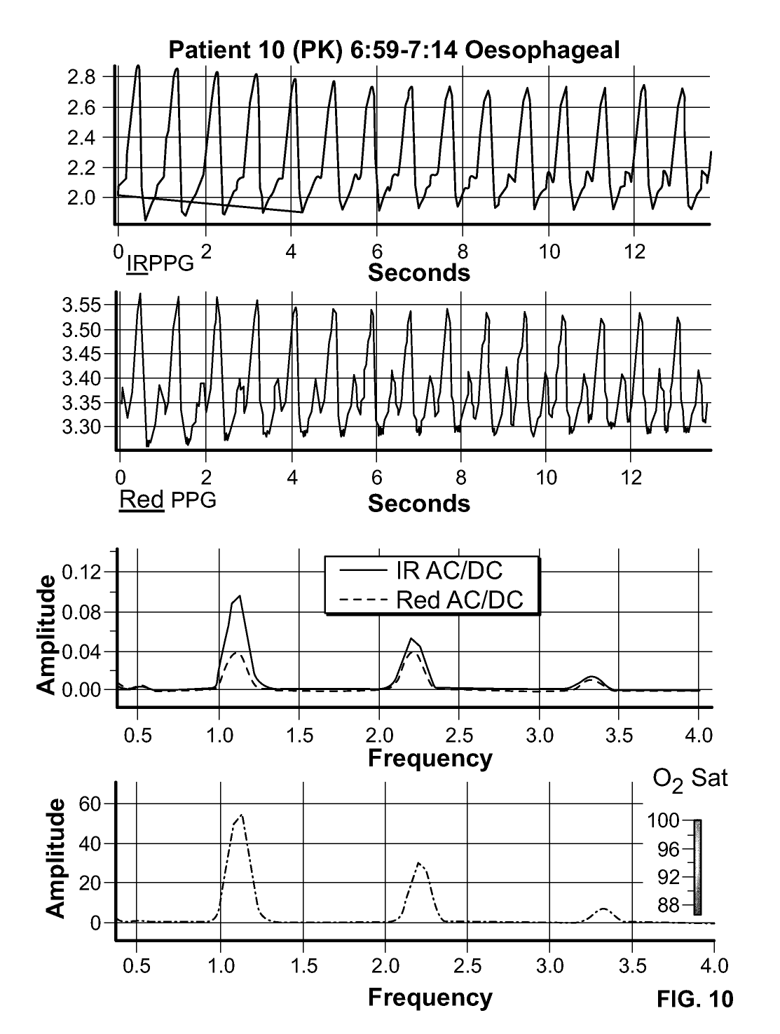

In exemplary embodiments, the determined instantaneous oxygen saturation may be overlaid relative to a time domain or frequency domain representation of the PG waveform (e.g. the PG waveform display may be color coded to indicate instantaneous oxygen saturation). Thus, venous oxygen saturation may be determined, e.g., by identifying a venous component/indicator for the PG waveform and reading the oxygen saturation corresponding to the venous component/indicator. Similarly, arterial oxygen may be determined, e.g., by identifying an arterial component/indicator for the PG waveform and reading the oxygen saturation corresponding to the arterial component/indicator. FIG. 10 depicts a frequency domain representation of exemplary Red/IR PG waveforms including an oxygen saturation overlay.

Consumption Detection

It is contemplated, that saturations other than oxygen saturation may also be detected by applying the present apparatus, systems and methods. Thus, e.g., glucose levels may be determined using a unique absorption signature characterized by absorption at a plurality of wavelengths, e.g., including infrared wavelengths. In accordance with the present apparatus, systems and methods, saturations, e.g., glucose saturation may advantageously be evaluated in two different regions of the vasculature (arterial and venous). Thus, in exemplary embodiments, consumption, e.g., glucose consumption, may be monitored based on the change in saturation from the arterial region to the venous region.

System Implementations

While in exemplary embodiments, the PG waveform, may be a obtained using photoplethysmograph (e.g., a pulse oximeter) it is appreciated that any of a number of known devices may be used to detect to the PG waveform. Accordingly, the present disclosure is not limited by the device used to obtain the PG waveform. Furthermore, while the present disclosure notes several exemplary measurement sites for obtaining the PG waveform (e.g., the ear, forehead, finger and esophagus), it is appreciated that any appropriate probe/measurement site for obtaining a PG waveform of the peripheral vasculature may be used. Accordingly, the present disclosure is not limited by the probe/measurement site used to obtain the PG waveform.

It is explicitly contemplated that the above methods may be carried out, e.g., via a processing unit having appropriate software, firmware and/or hardware. Thus, in exemplary embodiments, the plethysmograph device may include an interface for communicating with an external processing unit. The external processing unit may, for example, be a computer or other stand alone device having processing capabilities. Thus, in exemplary embodiments, the external processing unit may be a multifunction unit, e.g., with the ability to communicate with and process data for a plurality of measurement devices. Alternatively the plethysmograph device may include an internal or otherwise dedicated processing unit, typically a microprocessor or suitable logic circuitry. A plurality of processing units may, likewise, be employed. Thus, in exemplary embodiments, both dedicated and external processing units may be used.

The processing unit(s) of the present disclosure, generally, include means, e.g., hardware, firmware or software, for carrying out the above process of calibration/normalization. In exemplary embodiments, the hardware, firmware and/or software may be provided, e.g., as upgrade module(s) for use in conjunction with existing plethysmograph devices/processing units. Software/firmware may, e.g., advantageously include processable instructions, i.e. computer readable instructions, on a suitable storage medium for carrying out the above process. Similarly, hardware may, e.g., include components and/or logic circuitry for carrying out the above process.

A display and/or other feedback means may also be included to convey detected/processed data. Thus, in exemplary embodiments, oxygen saturation values, e.g., venous oxygen saturation and arterial oxygen saturation values, and/or other PG related data may be displayed, e.g., on a monitor. The display and/or other feedback means may be stand-alone or may be included as one or more components/modules of the processing unit(s) and/or plethysmograph device.

In general, it will be apparent to one of ordinary skill in the art that various embodiments described herein may be implemented in, or in association with, many different embodiments of software, firmware and/or hardware. The actual software code or specialized control hardware which may be used to implement some of the present embodiments is not intended to limit the scope of such embodiments. For example, certain aspects of the embodiments described herein may be implemented in computer software using any suitable computer software language type such as, for example, C or C++ using, for example, conventional or object-oriented techniques. Such software may be stored on any type of suitable computer-readable medium or media such as, for example, a magnetic or optical storage medium. Thus, the operation and behavior of the embodiments may be described without specific reference to the actual software code or specialized hardware components. The absence of such specific references is feasible because it is clearly understood that artisans of ordinary skill would be able to design software and control hardware to implement the various embodiments based on the description herein with only a reasonable effort and without undue experimentation.

Moreover, the methods of the present disclosure may be executed by, or in operative association with, programmable equipment, such as computers and computer systems. Software that cause programmable equipment to execute the methods may be stored in any storage device, such as, for example, a computer system (non-volatile) memory, an optical disk, magnetic tape, or magnetic disk. Furthermore, the processes may be programmed when the computer system is manufactured or via a computer-readable medium. Such a medium may include any suitable form.

It can also be appreciated that certain steps described herein may be performed using instructions stored on a computer-readable medium or media that direct a computer system to perform said steps. A computer-readable medium may include, for example, memory devices such as diskettes, compact discs of both read-only and read/write varieties, optical disk drives and hard disk drives. A computer-readable medium may also include memory storage that may be physical, virtual, permanent, temporary, semi-permanent and/or semi-temporary.

A “processor,” “processing unit,” “computer” or “computer system” may be, for example, a wireless or wireline variety of a microcomputer, minicomputer, server, mainframe, laptop, personal data assistant (PDA), wireless e-mail device (e.g., “BlackBerry” trade-designated devices), cellular phone, pager, processor, fax machine, scanner, or any other programmable device configured to transmit and receive data over a network. Computer systems disclosed herein may include memory for storing certain software applications used in obtaining, processing and communicating data. It can be appreciated that such memory may be internal or external to the disclosed embodiments. The memory may also include any means for storing software, including a hard disk, an optical disk, floppy disk, ROM (read only memory), RAM (random access memory), PROM (programmable ROM), EEPROM (electrically erasable PROM) and other computer-readable media.

Although the present disclosure has been described with reference to exemplary embodiments and implementations thereof, the disclosed systems, and methods are not limited to such exemplary embodiments/implementations. Rather, as will be readily apparent to persons skilled in the art from the description provided herein, the disclosed systems and methods are susceptible to modifications, alterations and enhancements without departing from the spirit or scope of the present disclosure. Accordingly, the present disclosure expressly encompasses such modification, alterations and enhancements within the scope hereof.

1. A method for determining saturation in a particular vascular region, the method comprising:
detecting a plethymograph (PG) waveform for each of a plurality of wavelengths;
calculating an instantaneous saturation for the detected PG waveforms;
extrapolating venous saturation or arterial saturation based on changes in the instantaneous saturation.

2. The method of claim 1, wherein venous saturation is extrapolated based on minimum instantaneous saturation values over a cardiac cycle.

3. The method of claim 1, wherein the calculating the instantaneous saturation comprises calculating, for each PG waveform, a delta AC value equal to an instantaneous value of an AC component of the PG waveform minus a corresponding minimum of the AC component, wherein the delta AC value is normalized by an offset of the PG waveform.

4. The method of claim 3, wherein if the normalized delta AC value is less than a threshold value, the normalized delta AC value is discarded and a prior normalized delta AC value is carried forward.

5. The method of claim 1, wherein the instantaneous oxygen saturation is overlaid on a time domain or frequency domain representation of one of the detected PG waveforms, wherein the venous saturation or arterial saturation is extrapolated based on instantaneous oxygen saturation values at venous or arterial indicators in the representation.
